(19) United States
(12) Patent Application Publication
(10) Pub. No.: US 2003/0017179 A1
(43) Pub. Date: Jan. 23, 2003

(54) Liquid oil-based cosmetic

(76) Inventor: Tetsuaki Akaishi, Takasaki-shi (JP)

Correspondence Address: Robert G. Mukai, BURNS, DOANE, SWECKER & MATHIS, L.L.P., P.O. Box 1404, Alexandria, VA, 22313-1404, US

(21) Appl. No.: 10/102,646

(22) Filed: Mar. 22, 2002

(30) Foreign Application Priority Data
Mar. 29, 2001 (JP) 2001-95941

(51) Int. Cl. A61K 7/00

(52) U.S. Cl. 424/401

(57) ABSTRACT

A liquid oil-based cosmetic used mainly for skin and nails, comprising a main ingredient of at least one kind of animal and vegetable oils, wherein panthenol having characteristics such as nutritional supply to skin and corneum is blended in animal and vegetable oils having blocking action in an amount of 0.4 to 10 wt % in a stable state together with 0.4 to 10 wt % of a glycol having an average molecular weight falling in a range of 90 to 400 and 5.0 to 40 wt % of a monohydric alcohol having an average molecular weight falling in a range of 110 to 250.

### BACKGROUND OF THE INVENTION

1. Field of the Invention

The present invention relates to a liquid oil-based cosmetic which can reveal an excellent skin chapping-prevention effect and corneum-improving effect and which is used mainly for skin and nails.

2. Description of the Related Art

Those called moisture cream, moisture lotion, emollient cream and emollient lotion have so far been used as cosmetics which can reveal skin chapping-prevention and corneum-improving effects. They are intended for preventing moisture from volatilizing (in general, called blocking action) by covering the surface of skin and corneum which are chapped by loss of moisture on the surface to hold moisture on the skin and corneum, and to soften and smoothen them. Mainly animal and vegetable oils are used for the ingredients thereof. Panthenol is a substance which has been blended at a low concentration in order to treat both hair and skin, and it is described in, for example, Japanese translations of PCT international Publication for Patent Application No. Hei 11-502872 that panthenol is blended with a vehicle in a range of preferably 1 wt % or less together with a polyalkylene glycol having n of 3 or more in the following formula to prepare a composition for treating hair or skin:

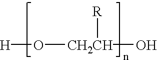

However, panthenol is water-soluble and therefore insoluble in animal and vegetable oils having blocking action of preventing moisture from volatilizing by covering skin and corneum as described above, and there has been the defect that even if it is compounded together with polyalkylene glycol described in Japanese Translations of PCT international Publication for Patent Application No. Hei 11-502872 described above, it is difficult to maintain a homogeneous dissolution state.

The present invention is intended for solving the problem described above, and an object thereof is to provide a liquid oil-based cosmetic which can reveal an excellent skin chapping-prevention effect and corneum-improving effect by blending animal and vegetable oils having blocking action with panthenol having characteristics such as nutritional supply particularly to skin and corneum in a stable state and which is used mainly for skin and nails.

### SUMMARY OF THE INVENTION

Intensive researches of the problem described above repeated by the present inventors have resulted in finding that panthenol can be present in a stable state by blending with animal and vegetable oils together with a glycol having a specific molecular weight and a monohydric alcohol having a high molecular weight, and thus the present invention has been completed.

That is, the present invention comprises the followings.

(1) A liquid oil-based cosmetic comprising a main ingredient of at least one kind of animal and vegetable oils having blocking action of preventing moisture from volatilizing by covering the surface of skin and corneum, further comprising 0.01 to 6.0 wt % of panthenol, 0.4 to 10 wt % of a glycol having an average molecular weight falling in a range of 90 to 400 and 5.0 to 40 wt % of a monohydric alcohol having an average molecular weight falling in a range of 110 to 250.

(2) The liquid oil-based cosmetic as described in the above item (1), wherein a blending weight ratio of the panthenol to the glycol having an average molecular weight falling in a range of 90 to 400 is 1:1 to 1:10.

(3) The liquid oil-based cosmetic as described in the above item 1 or (2), wherein a blending weight ratio of a mixture of the panthenol and the glycol having an average molecular weight falling in a range of 90 to 400 to the monohydric alcohol having an average molecular weight falling in a range of 110 to 250 is 1:1 to 1:40.

It is not certain how the liquid oil-based cosmetic in the present invention can reveal an excellent skin chapping-prevention effect and corneum-improving effect, but it is considered to result from the fact that panthenol having characteristics such as nutritional supply particularly to skin and corneum is contained in animal and vegetable oils having blocking action in a stable state by virtue of dissolving action brought about by use of the glycol in combination with the monohydric alcohol each described above.

### DETAILED DESCRIPTION OF THE PREFERED EMBODIMENTS

The embodiment of the present invention shall be explained below in details.

The liquid oil-based cosmetic of the present invention reveals an excellent skin chapping-prevention effect and corneum-improving effect by blending the panthenol, the glycol having an average molecular weight falling in a range of 90 to 400 and the monohydric alcohol having an average molecular weight falling in a range of 110 to 250 as essential blending ingredients each in a specific amount to the animal and vegetable oils having blocking action of preventing moisture from volatilizing by covering the surface of skin and corneum.

In this respect, the animal and vegetable oils having blocking action of preventing moisture from volatilizing by covering the surface of skin and corneum used in the present invention include beef tallow, lanolin, squalane, mink oil (all described above are animal oils), avocado oil, almond oil, olive oil, orange oil, sesame oil, corn oil, rape seed oil, castor oil, grape seed oil, jojoba oil, cotton seed oil, coconut oil, eucalyptus oil, peanut oil, rosemary oil and Roman chamomile oil (all described above are animal oils). In the present invention, at least one animal and vegetable oil selected from these groups is used.

The panthenol used in the present invention is alcohol corresponding to pantothenic acid and represented by a chemical structural formula of 2,4-dihydroxy-N-(3-hydroxypropyl)-3,3-dimethylbutaneamide. This substance is a viscous and hygroscopic liquid and readily soluble in water. It is easily turned into pantothenic acid invivo to take part in physiological action related to pantothenic acid. Present as stereoisomers in panthenol are, for example, a D(+) product (generally called D-pantothenyl alcohol) and an L(−) product, and particularly the D(+) product is considered to be a substance useful for treating skin and hair.

A blending proportion of the panthenol in the liquid oil-based cosmetic of the present invention is 0.01 to 6.0 wt %, preferably 0.05 to 5.0 wt % and more preferably 1 to 4 wt %. In this respect, if it is less than 0.01 wt %, the effect expected by adding panthenol is less liable to be obtained. On the other hand, if it exceeds 6.0 wt %, a sense of incompatibility is felt, and therefore such a range is not suitable.

Glycol having an average molecular weight falling in a range of 90 to 400 is used in the present invention. It includes, for example, a polyethylene glycol (average molecular weight: 200 to 400), diethylene glycol (molecular weight: 106), dipropylene glycol (molecular weight: 134) and 1,3-butylene glycol (molecular weight: 90).

If the average molecular weight falls in a range of 90 to 400, the glycol allows the panthenol to be improved in solubility in the animal and vegetable oils by using in combination with the monohydric alcohol described later, and the good stability in the dissolution state can be maintained well over a long period of time when the panthenol is blended with such animal and vegetable oils. If the glycol used has an average molecular weight of less than 90, the panthenol is well improved in solubility, but the stability shall be damaged when the panthenol is blended with animal and vegetable oils. On the other hand, if the glycol has an average molecular weight of exceeding 400, the panthenol is reduced in solubility in glycol, and therefore such a range is not preferred.

In the present invention, a weight ratio of the panthenol to the glycol having an average molecular weight falling in a range of 90 to 400 is preferably 1:1 to 1:10, more preferably 1:2 to 1:8. If the panthenol is added in excess of this weight ratio, the stability is damaged when blended with animal and vegetable oils. If the glycol is added in excess of the weight ratio described above, the characteristics of the panthenol and a blocking action endowed to the animal and vegetable oils are reduced. Accordingly, both ratios are not preferred.

The monohydric alcohol having an average molecular weight falling in a range of 110 to 250 is used in combination with the glycol in the present invention. The examples thereof include 1-dodecanol (lauryl alcohol: molecular weight 186), 2-ethylhexyl alcohol (molecular weight 130) and hexadecanol (molecular weight 242).

Addition of these monohydric alcohols makes it possible to allow the panthenol/glycol solution described above to be present in the animal and vegetable oils in a stable state over a long period of time. In the present invention, a blending amount of the monohydric alcohol is 5 to 40 wt %, preferably 8 to 15 wt %. If the blending amount is less than 5 wt %, the homogeneous dissolution state can not be maintained when the panthenol/glycol solution is blended with the animal and vegetable oils, and the stability is damaged. On the other hand, if the blending amount is 40 wt % or more, a blocking action endowed to the animal and vegetable oils is reduced. Accordingly, both ranges are not preferred.

In the present invention, a blending weight ratio of the panthenol/glycol solution described above to the monohydric alcohol having an average molecular weight falling in a range of 110 to 250 shall not specifically be restricted and is 1:1 to 1:40, preferably 1:2 to 1:10. If the panthenol/glycol solution is added in excess of this weight ratio, the stability is liable to be damaged when blended with the animal and vegetable oils. If the monohydric alcohol is added in excess of this weight ratio described above, the panthenol and the animal and vegetable oils are decreased in blending proportion, and the function as a cosmetic is liable to be reduced. Accordingly, both rations are not preferred.

It is no problem that the liquid oil-based cosmetic of the present invention is optionally blended, in addition thereto, with a known polyol as a moisture holding component, a natural moisture holding component, vitamins, various amino acids and preservatives, a UV absorber and an antioxidant.

In order to obtain the liquid oil-based cosmetic of the present invention, the panthenol, the glycol and the monohydric alcohol each described above are weighed in advance in prescribed amounts and blended, and they are heated at 100° C. or lower, preferably about 60° C. for about one hour and dissolved. The solution is stirred and mixed by means of a stirrer such as a disper until the solution is homogenized, and then it is cooled down. Thereafter, a main component of the animal and vegetable oils and the other optional components are added, and they are stirred and evenly mixed by means of a disper, whereby the liquid oil-based cosmetic of the present invention can be produced.

The specific uses of the liquid oil-based cosmetic of the present invention include moisture cream, moisture lotion, emollient cream and emollient lotion in which it is expected that mainly skin chapping-prevention and corneum-improving effects can be revealed.

### EXAMPLES

The present invention shall be explained below in further details with reference to examples and comparative examples, but the present invention shall by no means be restricted by these examples.

Stability tests and sensory evaluations in actual uses in the following examples and comparative examples were judged in the following manners.

(1) Stability Test:

The respective samples prepared were left standing in a sample bottle for one week and one month, and then the dissolution state of the panthenol was visually observed:

∘: sample is clear

Δ: sample is observed to be a little turbid or tend to be slightly separated into phases

×: sample is observed to tend to be completely separated into phases

(2) Sensory Evaluation in Actual Uses:

The respective samples prepared were coated once a day by monitors always using a manicure on their nails and peripheral parts thereof after removing the manicure to sensuously evaluate the state of the nails and the peripheral part thereof after used for a fixed time according to the following criteria:

⊚: dryness and hangnail on the surface of a nail were improved to a large extent about two weeks after used

∘: dryness and hangnail on the surface of a nail were observed to tend to be improved about one month after used

Δ: dryness and hangnail on the surface of a nail were observed to tend to be improved one month or longer after used

×: dryness and hangnail on the surface of a nail were not observed to be improved

### Example 1

Among components of the following blending rates (wt %), the components (1) to (3) were heated at 60° C. for one hour and then stirred by means of a disper until the solution was homogenized. After cooling down, the remaining components (4) to (8) were added and stirred by means of the disper until the solution was homogenized, whereby a cosmetic sample of the present invention was obtained.

| | | |
|---|---|---|
| (1) | Panthenol (D-pantothenyl alcohol) | 2.0 wt % |
| (2) | Polyethylene glycol (average molecular | 8.0 wt % |
| | weight: 400) | |
| (3) | 1-Dodecanol (molecular weight: 186) | 30.0 wt % |
| (4) | Isopropyl palmitate (moisture holding agent) | 10.0 wt % |
| (5) | Di(phytostery.2-octyldodecyl) N-lauroyl- | 8.0 wt % |
| | L-glutamate (moisture holding agent) | |
| (6) | Paraben (paraoxybenzoic acid ester: | 0.2 wt % |
| | preservative) | |
| (7) | Dibutylhydroxytoluene (antioxidant) | 0.1 wt % |
| (8) | Safflower oil (blocking action) | 41.7 wt % |

### Example 2

The components were stirred at the same blending rates as in Example 1 under the same conditions by means of the disper until the solution was homogenized to obtain a cosmetic sample of the present invention, except that (2) polyethylene glycol in Example 1 was changed to diethylene glycol (molecular weight: 106).

### Example 3

The components were stirred at the same blending rates as in Example 1 under the same conditions by means of the disper until the solution was homogenized to obtain a cosmetic sample of the present invention, except that (2) polyethylene glycol in Example 1 was changed to dipropylene glycol (molecular weight: 134).

### Example 4

The components were stirred at the same blending rates as in Example 1 under the same conditions by means of the disper until the solution was homogenized to obtain a cosmetic sample of the present invention, except that (2) polyethylene glycol in Example 1 was changed to 1,3-butylene glycol (molecular weight: 90).

### Example 5

The components were stirred at the same blending rates as in Example 1 under the same conditions by means of the disper until the solution was homogenized to obtain a cosmetic sample of the present invention, except that (3) 1-dodecanol in Example 1 was changed to 2-ethylhexyl alcohol (molecular weight: 130).

### Example 6

The components were stirred at the same blending rates as in Example 2 under the same conditions by means of the disper until the solution was homogenized to obtain a cosmetic sample of the present invention, except that (3) 1-dodecanol in Example 2 was changed to 2-ethylhexyl alcohol (molecular weight: 130).

### Example 7

The components were stirred at the same blending rates as in Example 3 under the same conditions by means of the disper until the solution was homogenized to obtain a cosmetic sample of the present invention, except that (3) 1-dodecanol in Example 3 was changed to 2-ethylhexyl alcohol (molecular weight: 130).

### Example 8

The components were stirred at the same blending rates as in Example 4 under the same conditions by means of the disper until the solution was homogenized to obtain a cosmetic sample of the present invention, except that (3) 1-dodecanol in Example 4 was changed to 2-ethylhexyl alcohol (molecular weight: 130).

### Example 9

The components were stirred at the same blending rates as in Example 1 under the same conditions by means of the disper until the solution was homogenized to obtain a cosmetic sample of the present invention, except that a blending rate of (3) 1-dodecanol in Example 1 was changed from 30.0 wt % to 20.0 wt % and a blending rate of (8) Safflower oil (blocking action) was changed from 41.7 wt % to 51.7 wt %.

### Example 10

The components were stirred at the same blending rates as in Example 1 under the same conditions by means of the disper until the solution was homogenized to obtain a cosmetic sample of the present invention, except that a blending rate of (3) 1-dodecanol in Example 1 was changed from 30.0 wt % to 10.0 wt % and a blending rate of (8) Safflower oil (blocking action) was changed from 41.7 wt % to 61.7 wt %.

### Example 11

The components were stirred at the same blending rates as in Example 1 under the same conditions by means of the disper until the solution was homogenized to obtain a cosmetic sample of the present invention, except that a blending rate of (1) panthenol in Example 1 was changed from 2 wt % to 1 wt % and a blending rate of (2) polyethylene glycol was changed from 8.0 wt % to 10.0 wt % and that a blending rate of (8) Safflower oil was changed from 41.7 wt % to 40.7 wt %.

### Example 12

The components were stirred at the same blending rates as in Example 1 under the same conditions by means of the disper until the solution was homogenized to obtain a cosmetic sample of the present invention, except that a blending rate of (1) panthenol in Example 1 was changed from 2 wt % to 5 wt % and a blending rate of (2) polyethylene glycol was changed from 8.0 wt % to 10.0 wt % and that a blending rate of (8) Safflower oil was changed from 41.7 wt % to 36.7 wt %.

### Example 13

The components were stirred at the same blending rates as in Example 3 under the same conditions by means of the disper until the solution was homogenized to obtain a cosmetic sample of the present invention, except that (8) Safflower oil (blocking action) in Example 3 was changed to squalane.

### Example 14

The components were stirred at the same blending rates as in Example 4 under the same conditions by means of the disper until the solution was homogenized to obtain a cosmetic sample of the present invention, except that (8) Safflower oil (blocking action) in Example 4 was changed to squalane.

### Example 15

The components were stirred at the same blending rates as in Example 13 under the same conditions by means of the disper until the solution was homogenized to obtain a cosmetic sample of the present invention, except that only (3) 1-dodecanol in Example 13 was changed to 2-ethylhexyl alcohol (molecular weight: 130).

### Example 16

The components were stirred at the same blending rates as in Example 14 under the same conditions by means of the disper until the solution was homogenized to obtain a cosmetic sample of the present invention, except that (3) 1-dodecanol in Example 14 was changed to 2-ethylhexyl alcohol (molecular weight: 130).

### Example 17

The components were stirred at the same blending rates as in Example 13 under the same conditions by means of the disper until the solution was homogenized to obtain a cosmetic sample of the present invention, except that only (3) 1-dodecanol in Example 13 was changed to hexadecanol (molecular weight: 242).

### Comparative Example 1

The components were stirred at the same blending rates as in Example 1 under the same conditions by means of the disper until the solution was homogenized to obtain a comparative sample, except that (2) polyethylene glycol (average molecular weight: 400) in Example 1 was changed to polyethylene glycol (average molecular weight: 600).

### Comparative Example 2

The components were stirred at the same blending rates as in Example 5 under the same conditions by means of the disper until the solution was homogenized to obtain a comparative sample, except that (2) polyethylene glycol (average molecular weight: 400) in Example 5 was changed to polyethylene glycol (average molecular weight: 600).

### Comparative Example 3

The components were stirred at the same blending rates as in Example 1 under the same conditions by means of the disper until the solution was homogenized to obtain a comparative sample, except that (3) 1-dodecanol in Example 1 was changed to benzyl alcohol (molecular weight: 108).

### Comparative Example 4

The components were stirred at the same blending rates as in Example 2 under the same conditions by means of the disper until the solution was homogenized to obtain a comparative sample, except that (3) 1-dodecanol in Example 2 was changed to benzyl alcohol (molecular weight: 108).

### Comparative Example 5

The components were stirred at the same blending rates as in Example 1 under the same conditions by means of the disper until the solution was homogenized to obtain a comparative sample, except that (2) polyethylene glycol in Example 1 was changed to propylene glycol (molecular weight: 75) and (3) 1-dodecanol was changed to benzyl alcohol (molecular weight: 108).

### Comparative Example 6

The components were stirred at the same blending rates as in Example 3 under the same conditions by means of the disper until the solution was homogenized to obtain a comparative sample, except that (3) 1-dodecanol in Example 3 was changed to benzyl alcohol (molecular weight: 108).

### Comparative Example 7

The components were stirred at the same blending rates as in Example 4 under the same conditions by means of the disper until the solution was homogenized to obtain a comparative sample, except that (3) 1-dodecanol in Example 4 was changed to benzyl alcohol (molecular weight: 108).

### Comparative Example 8

The components were stirred at the same blending rates as in Example 1 under the same conditions by means of the disper until the solution was homogenized to obtain a comparative sample, except that (3) 1-dodecanol in Example 1 was changed to oleyl alcohol (molecular weight: 268).

### Comparative Example 9

The components were stirred at the same blending rates as in Example 2 under the same conditions by means of the disper until the solution was homogenized to obtain a comparative sample, except that (3) 1-dodecanol in Example 2 was changed to oleyl alcohol (molecular weight: 268).

### Comparative Example 10

The components were stirred at the same blending rates as in Example 1 under the same conditions by means of the disper until the solution was homogenized to obtain a comparative sample, except that (2) polyethylene glycol in Example 1 was changed to propylene glycol (molecular weight: 75) and (3) 1-dodecanol was changed to oleyl alcohol (molecular weight: 268).

### Comparative Example 11

The components were stirred at the same blending rates as in Example 3 under the same conditions by means of the disper until the solution was homogenized to obtain a comparative sample, except that (3) 1-dodecanol in Example 3 was changed to oleyl alcohol (molecular weight: 268).

### Comparative Example 12

The components were stirred at the same blending rates as in Example 4 under the same conditions by means of the disper until the solution was homogenized to obtain a comparative sample, except that (3) 1-dodecanol in Example 4 was changed to oleyl alcohol (molecular weight: 268).

### Comparative Example 13

The components were stirred at the same blending rates as in Example 13 under the same conditions by means of the disper until the solution was homogenized to obtain a comparative sample, except that (3) 1-dodecanol in Example 13 was changed to benzyl alcohol (molecular weight: 108).

### Comparative Example 14

The components were stirred at the same blending rates as in Example 14 under the same conditions by means of the disper until the solution was homogenized to obtain a comparative sample, except that (3) 1-dodecanol in Example 14 was changed to benzyl alcohol (molecular weight: 108).

### Comparative Example 15

The components were stirred at the same blending rates as in Example 15 under the same conditions by means of the disper until the solution was homogenized to obtain a comparative sample, except that 2-ethylhexyl alcohol in Example 15 was changed to oleyl alcohol (molecular weight: 268).

### Comparative Example 16

The components were stirred at the same blending rates as in Example 16 under the same conditions by means of the disper until the solution was homogenized to obtain a comparative sample, except that 2-ethylhexyl alcohol in Example 16 was changed to oleyl alcohol (molecular weight: 268).

Shown together in Tables 1 and 2 are the main components and the blending rates in Examples 1 to 17 and Comparative Examples 1 to 16 and the respective test results of the cosmetic samples of the present invention and the comparative samples obtained.

TABLE 1

| Component/Example No. | No. 1 | No. 2 | No. 3 | No. 4 | No. 5 | No. 6 | No. 7 | No. 8 | No. 9 |
|---|---|---|---|---|---|---|---|---|---|
| Pantenol | 2.0 | 2.0 | 2.0 | 2.0 | 2.0 | 2.0 | 2.0 | 2.0 | 2.0 |
| Polyethylene glycol 400 | 8.0 | | | | 8.0 | | | | 8.0 |
| Diethylene glycol | | 8.0 | | | | 8.0 | | | |
| Propylene glycol | | | | | | | | | |
| Dipropylene glycol | | | 8.0 | | | | 8.0 | | |
| 1,3-Butylene glycol | | | | 8.0 | | | | 8.0 | |
| 1-Dodecanol | 30.0 | 30.0 | 30.0 | 30.0 | | | | | 20.0 |
| 2-Ethylhexyl alcohol | | | | | 30.0 | 30.0 | 30.0 | 30.0 | |
| Hexadecanol | | | | | | | | | |
| Isopropyl palmitate | 10.0 | 10.0 | 10.0 | 10.0 | 10.0 | 10.0 | 10.0 | 10.0 | 10.0 |
| Di(phytostery.2-octyldodecy) N- | 8.0 | 8.0 | 8.0 | 8.0 | 8.0 | 8.0 | 8.0 | 8.0 | 8.0 |
| lauroyl-L-glutamate | | | | | | | | | |
| Paraben | 0.2 | 0.2 | 0.2 | 0.2 | 0.2 | 0.2 | 0.2 | 0.2 | 0.2 |
| Dibutylhydroxytoluene | 0.1 | 0.1 | 0.1 | 0.1 | 0.1 | 0.1 | 0.1 | 0.1 | 0.1 |
| Safflower oil | 41.7 | 41.7 | 41.7 | 41.7 | 41.7 | 41.7 | 41.7 | 41.7 | 51.7 |
| Squalane | | | | | | | | | |
| Polyethylene glycol 600 | | | | | | | | | |
| Benzyl alcohol | | | | | | | | | |
| Oleyl alcohol | | | | | | | | | |
| (Total) | 100.0 | 100.0 | 100.0 | 100.0 | 100.0 | 100.0 | 100.0 | 100.0 | 100.0 |
| Test results | | | | | | | | | |
| Stability test (after one week) | ◯ | ◯ | ◯ | ◯ | ◯ | ◯ | ◯ | ◯ | ◯ |
| Stability test (after one month) | ◯ | ◯ | ◯ | ◯ | ◯ | ◯ | ◯ | ◯ | ◯ |
| Actual use functional test | ◯ | ◯ | ◯ | ◯ | ◯ | ◯ | ◯ | ◯ | ◯ |

| Component/Example No. | No. 10 | No. 11 | No. 12 | No. 13 | No. 14 | No. 15 | No. 16 | No. 17 |
|---|---|---|---|---|---|---|---|---|
| Pantenol | 2.0 | 2.0 | 5.0 | 2.0 | 2.0 | 2.0 | 2.0 | 2.0 |
| Polyethylene glycol 400 | 8.0 | 10.0 | 10.0 | | | | | |
| Diethylene glycol | | | | | | | | |
| Propylene glycol | | | | | | | | |
| Dipropylene glycol | | | | 8.0 | | 8.0 | | 8.0 |
| 1,3-Butylene glycol | | | | | 8.0 | | 8.0 | |
| 1-Dodecanol | 10.0 | 30.0 | 30.0 | 30.0 | 30.0 | | | |
| 2-Ethylhexyl alcohol | | | | | | 30.0 | 30.0 | |
| Hexadecanol | | | | | | | | 30.0 |
| Isopropyl palmitate | 10.0 | 10.0 | 10.0 | 10.0 | 10.0 | 10.0 | 10.0 | 10.0 |
| Di(phytostery.2-octyldodecy) N- | 8.0 | 8.0 | 8.0 | 8.0 | 8.0 | 8.0 | 8.0 | 8.0 |
| lauroyl-L-glutamate | | | | | | | | |
| Paraben | 0.2 | 0.2 | 0.2 | 0.2 | 0.2 | 0.2 | 0.2 | 0.2 |
| Dibutylhydroxytoluene | 0.1 | 0.1 | 0.1 | 0.1 | 0.1 | 0.1 | 0.1 | 0.1 |
| Safflower oil | 61.7 | 40.7 | 36.7 | | | | | |
| Squalane | | | | 41.7 | 41.7 | 41.7 | 41.7 | 41.7 |
| Polyethylene glycol 600 | | | | | | | | |
| Benzyl alcohol | | | | | | | | |
| Oleyl alcohol | | | | | | | | |
| (Total) | 100.0 | 100.0 | 100.0 | 100.0 | 100.0 | 100.0 | 100.0 | 100.0 |
| Test results | | | | | | | | |
| Stability test (after one week) | ◯ | ◯ | ◯ | ◯ | ◯ | ◯ | ◯ | ◯ |
| Stability test (after one month) | ◯ | ◯ | ◯ | ◯ | ◯ | ◯ | ◯ | ◯ |
| Actual use functional test | ⊚ | ◯ | ◯ | ◯ | ◯ | ◯ | ◯ | ⊚ |

TABLE 2

| Component/Comparative Example No. | No. 1 | No. 2 | No. 3 | No. 4 | No. 5 | No. 6 | No. 7 | No. 8 |
|---|---|---|---|---|---|---|---|---|
| Pantenol | 2.0 | 2.0 | 2.0 | 2.0 | 2.0 | 2.0 | 2.0 | 2.0 |
| Polyethylene glycol 400 | | | 8.0 | | | | | 8.0 |
| Diethylene glycol | | | | 8.0 | | | | |
| Propylene glycol | | | | | 8.0 | | | |
| Dipropylene glycol | | | | | | 8.0 | | |
| 1,3-Butylene glycol | | | | | | | 8.0 | |
| 1-Dodecanol | 30.0 | | | | | | | |
| 2-Ethylhexyl alcohol | | 30.0 | | | | | | |
| Hexadecanol | | | | | | | | |
| Isopropyl palmitate | 10.0 | 10.0 | 10.0 | 10.0 | 10.0 | 10.0 | 10.0 | 10.0 |
| Di(phytostery.2-octyldodecy) N- | 8.0 | 8.0 | 8.0 | 8.0 | 8.0 | 8.0 | 8.0 | 8.0 |
| lauroyl-L-glutamate | | | | | | | | |
| Paraben | 0.2 | 0.2 | 0.2 | 0.2 | 0.2 | 0.2 | 0.2 | 0.2 |
| Dibutylhydroxytoluene | 0.1 | 0.1 | 0.1 | 0.1 | 0.1 | 0.1 | 0.1 | 0.1 |
| Safflower oil | 41.7 | 41.7 | 41.7 | 41.7 | 41.7 | 41.7 | 41.7 | 41.7 |
| Squalane | | | | | | | | |
| Polyethylene glycol 600 | 8.0 | 8.0 | | | | | | |
| Benzyl alcohol | | | 30.0 | 30.0 | 30.0 | 30.0 | 30.0 | |
| Oleyl alcohol | | | | | | | | 30.0 |
| (Total) | 100.0 | 100.0 | 100.0 | 100.0 | 100.0 | 100.0 | 100.0 | 100.0 |
| Test results | | | | | | | | |
| Stability test (after one week) | X | X | X | X | X | X | X | Δ |
| Stability test (after one month) | X | X | X | X | X | X | X | X |
| Actual use functional test | Δ˜X | Δ˜X | Δ˜X | Δ˜X | Δ˜X | Δ˜X | Δ˜X | Δ |
| Component/Comparative Example No. | No. 9 | No. 10 | No. 11 | No. 12 | No. 13 | No. 14 | No. 15 | No. 16 |
| Pantenol | 2.0 | 2.0 | 2.0 | 2.0 | 2.0 | 2.0 | 2.0 | 2.0 |
| Polyethylene glycol 400 | | | | | | | | |
| Diethylene glycol | 8.0 | | | | | | | |
| Propylene glycol | | 8.0 | | | | | | |
| Dipropylene glycol | | | 8.0 | | 8.0 | | 8.0 | |
| 1,3-Butylene glycol | | | | 8.0 | | 8.0 | | 8.0 |
| 1-Dodecanol | | | | | | | | |
| 2-Ethylhexyl alcohol | | | | | | | | |
| Hexadecanol | | | | | | | | |
| Isopropyl palmitate | 10.0 | 10.0 | 10.0 | 10.0 | 10.0 | 10.0 | 10.0 | 10.0 |
| Di(phytostery.2-octyldodecy) N- | 8.0 | 8.0 | 8.0 | 8.0 | 8.0 | 8.0 | 8.0 | 8.0 |
| lauroyl-L-glutamate | | | | | | | | |
| Paraben | 0.2 | 0.2 | 0.2 | 0.2 | 0.2 | 0.2 | 0.2 | 0.2 |
| Dibutylhydroxytoluene | 0.1 | 0.1 | 0.1 | 0.1 | 0.1 | 0.1 | 0.1 | 0.1 |
| Safflower oil | 41.7 | 41.7 | 41.7 | 41.7 | | | | |
| Squalane | | | | | 41.7 | 41.7 | 41.7 | 41.7 |
| Polyethylene glycol 600 | | | | | | | | |
| Benzyl alcohol | | | | | 30.0 | 30.0 | | |
| Oleyl alcohol | 30.0 | 30.0 | 30.0 | 30.0 | | | 30.0 | 30.0 |
| (Total) | 100.0 | 100.0 | 100.0 | 100.0 | 100.0 | 100.0 | 100.0 | 100.0 |
| Test results | | | | | | | | |
| Stability test (after one week) | Δ | Δ | ◯ | Δ | X | X | Δ | Δ |
| Stability test (after one month) | X | X | Δ | X | X | X | X | X |
| Actual use functional test | Δ | Δ | Δ | Δ | Δ | Δ | Δ | Δ |

As apparent from the results shown in Tables 1 and 2, it has been found that according to the present invention, blending of glycol having an average molecular weight falling in a range of 90 to 400 and monohydric alcohol having an average molecular weight falling in a range of 110 to 250 allows panthenol to be present in animal and vegetable oils in a stable state and provides a liquid oil-based cosmetic which can reveal an excellent skin chapping-prevention effect and corneum-improving effect and which is used mainly for skin and nails.

What is claimed is:

1. A liquid oil-based cosmetic comprising a main ingredient of at least one kind of animal and vegetable oils having blocking action of preventing moisture from volatilizing by covering the surface of skin and corneum, further comprising 0.01 to 6.0 wt % of panthenol, 0.4 to 10 wt % of a glycol having an average molecular weight falling in a range of 90 to 400 and 5.0 to 40 wt % of a monohydric alcohol having an average molecular weight falling in a range of 110 to 250.

2. The liquid oil-based cosmetic as described in claim 1, wherein a blending weight ratio of the panthenol to the glycol having an average molecular weight falling in a range of 90 to 400 is 1:1 to 1:10.

3. The liquid oil-based cosmetic as described in claim 1 or 2, wherein a blending weight ratio of a mixture of the panthenol and the glycol having an average molecular weight falling in a range of 90 to 400 to the monohydric alcohol having an average molecular weight falling in a range of 110 to 250 is 1:1 to 1:40.
